(19) United States
(12) Patent Application Publication
(10) Pub. No.: US 2026/0364189 A1
(43) Pub. Date: Nov. 19, 2026

(54) ANTI-TFR1 ANTIBODIES AND USES THEREOF

(71) Applicant: Biocytogen Pharmaceuticals (Beijing) Co., Ltd., Beijing (CN)

(72) Inventors: Yiqing Hu, Beijing (CN); Lijun Zhang, Beijing (CN); Huizhen Zhao, Beijing (CN); Jiawei Yao, Beijing (CN); Yuelei Shen, Beijing (CN)

(21) Appl. No.: 19/133,797

(22) PCT Filed: Nov. 29, 2023
(86) PCT No.: PCT/CN2023/135130

§ 371 Date: May 29, 2025

(30) Foreign Application Priority Data
Dec. 2, 2022 (WO) PCT/CN2022/136246

(51) Int. Cl. A61K 47/68; A61P 35/00; C07K 16/18; C07K 16/28; G01N 33/575

(52) U.S. Cl. CPC A61K 47/68037; A61K 47/6849; A61P 35/00; C07K 16/18; C07K 16/2881; G01N 33/57557; G01N 33/5759; C07K 2317/14; C07K 2317/24; C07K 2317/31; C07K 2317/52; C07K 2317/565; C07K 2317/569; C07K 2317/76; G01N 2333/70582

(57) ABSTRACT

The present disclosure provides an anti-TFR1 (transferrin receptor 1) antibodies, antigen-binding fragments, and the uses thereof.

### CLAIM OF PRIORITY

This application claims the benefit of PCT Application No. PCT/CN2022/136246, filed on Dec. 2, 2022. The entire contents of the foregoing are incorporated herein by reference.

### TECHNICAL FIELD

This disclosure relates to anti-TFR1 (transferrin receptor 1) antibodies, antigen-binding fragments, and the uses thereof.

### BACKGROUND

Therapeutic antibodies are one of the fastest growing classes of therapeutic compounds, rapidly outpacing the growth of small-molecule drugs. For example, monoclonal antibodies have revolutionized cancer therapy. However, delivery to tumor cells in vivo is hampered by the large size of conventional antibodies. The minimal target recognition module of a conventional antibody is composed of two non-covalently associated variable domains (VH and VL). The inherent hydrophobic interaction of VH and VL domains limits the stability and solubility of engineered antibodies, often causing aggregation and/or mispairing of V-domains.

The discovery of heavy-chain antibodies has given rise to unprecedented opportunities in impacting cancer therapy. These unique forms of camelid-derived antibodies lack the entire light chain and the CH1 domain and are only composed of a single variable domain termed VHH. Recombinant VHHs are small (15-20 kDa) and strictly monomeric; they bind their target with nM affinity as well as with being stable in a broad pH and temperature ranges. Molecular manipulation is also easier with VHH; this facilitates the production of multivalent formats of monoclonal antibodies compared with conventional recombinant antibodies and their fragments, which is problematic due to aggregation and reduced affinity. Moreover, VHH often binds to epitopes that are less immunogenic for conventional antibodies.

Usually, the therapeutic antibodies are human or humanized antibodies. The human or humanized antibodies can be generated by humanization of a rodent antibody (e.g., a mouse antibody) or by using phage libraries. However, these animals or phage libraries usually cannot produce heavy chain antibodies. Instead, heavy chain antibodies are often obtained from camelid heavy chain antibodies. These camelid heavy chain antibodies need to be humanized. The humanization process may adversely affect the binding affinity and introduce immunogenic epitopes to the antibodies. Iterative and time-consuming experiments are often required to improve the properties of these antibodies. And in some cases, these antibodies can also be immunogenic in patients, leading to attenuation of their efficacy over time. Therefore, there is a need to develop more forms of antibodies to treat or prevent human diseases.

### SUMMARY

The present disclosure relates to an antibody or antigen-binding fragment thereof that binds to transferrin receptor 1 (TFR1), comprising: a heavy chain single variable domain (VHH) comprising complementarity determining regions (CDRs) 1, 2, and 3, in some embodiments, the VHH CDR1 region comprises an amino acid sequence that is at least 80%, 85%, 90%, 95%, 96%, 97%, 98%, 99%, or 100% identical to a selected VHH CDR1 amino acid sequence, the VHH CDR2 region comprises an amino acid sequence that is at least 80%, 85%, 90%, 95%, 96%, 97%, 98%, 99%, or 100% identical to a selected VHH CDR2 amino acid sequence, and the VHH CDR3 region comprises an amino acid sequence that is at least 80%, 85%, 90%, 95%, 96%, 97%, 98%, 99%, or 100% identical to a selected VHH CDR3 amino acid sequence; in some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively;
(4) the selected VHH CDRs 1, 2, 3 amino acid sequences are set forth in SEQ ID NOs: 10, 11, and 12, respectively;
(5) the selected VHH CDRs 1, 2, 3 amino acid sequences are set forth in SEQ ID NOs: 13, 14, and 15, respectively;
(6) the selected VHH CDRs 1, 2, 3 amino acid sequences are set forth in SEQ ID NOs: 16, 17, and 18, respectively;
(7) the selected VHH CDRs 1, 2, 3 amino acid sequences are set forth in SEQ ID NOs: 19, 20, and 21, respectively; and
(8) the selected VHH CDRs 1, 2, 3 amino acid sequences are set forth in SEQ ID NOs: 22, 23, and 24, respectively.

In some embodiments, the VHH comprises CDRs 1, 2, 3 with the amino acid sequences set forth in SEQ ID NOs: 1, 2, and 3, respectively. In some embodiments, the VHH comprises CDRs 1, 2, 3 with the amino acid sequences set forth in SEQ ID NOs: 4, 5, and 6, respectively. In some embodiments, the VHH comprises CDRs 1, 2, 3 with the amino acid sequences set forth in SEQ ID NOs: 7, 8, and 9, respectively. In some embodiments, the VHH comprises CDRs 1, 2, 3 with the amino acid sequences set forth in SEQ ID NOs: 10, 11, and 12, respectively.

In one aspect, the disclosure is related to an antibody or antigen-binding fragment thereof that binds to TFR1 comprising a heavy chain single variable region (VHH) comprising an amino acid sequence that is at least 80%, 85%, 90%, 95%, 96%, 97%, 98%, 99%, or 100% identical to a selected VHH sequence, in some embodiments, the selected VHH sequence is selected from the group consisting of SEQ ID NOs: 25, 26, 27, and 28. In some embodiments, the VHH comprises the sequence of SEQ ID NO: 25. In some embodiments, the VHH comprises the sequence of SEQ ID NO: 26. In some embodiments, the VHH comprises the sequence of SEQ ID NO: 27. In some embodiments, the VHH comprises the sequence of SEQ ID NO: 28. In some embodiments, the antibody or antigen-binding fragment specifically binds to a human TFR1, a monkey TFR1, a mouse TFR1, or a chimeric TFR1. In some embodiments, the antibody or antigen-binding fragment is a human or humanized antibody or antigen-binding fragment thereof. In some embodiments, the antibody or antigen-binding fragment is a multi-specific antibody (e.g., a bispecific antibody).

In one aspect, the disclosure is related to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc (e.g., a human IgG1 Fc). In some embodiments, the human IgG Fc comprises a non-asparagine residue (e.g., alanine) at position 297 according to EU numbering. In some embodiments, the antibody or antigen-binding fragment comprises two or more heavy chain single variable domains.

In one aspect, the disclosure is related to a nucleic acid comprising a polynucleotide encoding the antibody or antigen-binding fragment thereof as described herein. In some embodiments, the nucleic acid is cDNA.

In one aspect, the disclosure is related to a vector comprising one or more of the nucleic acids as described herein.

In one aspect, the disclosure is related to a cell comprising the vector as described herein. In some embodiments, the cell is a CHO cell. In one aspect, the disclosure is related to a cell comprising one or more of the nucleic acids described herein.

In one aspect, the disclosure is related to a method of producing an antibody or an antigen-binding fragment thereof, the method comprising (a) culturing the cell as described herein under conditions sufficient for the cell to produce the antibody or the antigen-binding fragment thereof; and (b) collecting the antibody or the antigen-binding fragment thereof produced by the cell.

In one aspect, the disclosure is related to an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure is related to a method of treating a subject having a brain disease (e.g., a brain cancer), the method comprising administering a therapeutically effective amount of a composition comprising the antibody or antigen-binding fragment thereof, or the antibody-drug conjugate as described herein, to the subject. In some embodiments, the antibody or antigen-binding fragment thereof, or the antibody-drug conjugate can pass across the blood-brain barrier (BBB) of the subject.

In one aspect, the disclosure is related to a method of treating a subject having a cancer, the method comprising administering a therapeutically effective amount of a composition comprising the antibody or antigen-binding fragment thereof, or the antibody-drug conjugate as described herein, to the subject. In some embodiments, the cancer is brain cancer, lung cancer, gastric cancer, colorectal cancer, liver cancer, ovarian cancer, prostate cancer, leukemia, or breast cancer. In one aspect, the disclosure is related to a method of identifying a subject as having a brain disease (e.g., a brain cancer), the method comprising detecting a sample collected from the subject as having the brain disease by the antibody or antigen-binding fragment thereof as described herein, thereby identifying the subject as having the brain disease. In some embodiments, the sample is a brain parenchyma sample from the subject. In some embodiments, the subject described herein is a human subject.

In one aspect, the disclosure is related to a method of delivering an agent to cross blood brain barrier, the method comprising administering the agent covalently linked to the antibody or antigen-binding fragment thereof as described herein to the subject. In some embodiments, the agent is an antibody or an antibody drug conjugate. In some embodiments, the agent is anti-amyloid antibody.

In one aspect, the disclosure is related to a pharmaceutical composition comprising the antibody or antigen-binding fragment thereof as described herein, and a pharmaceutically acceptable carrier. In one aspect, the disclosure is related to a pharmaceutical composition comprising the antibody drug conjugate as described herein, and a pharmaceutically acceptable carrier.

In one aspect, the disclosure is related to an antibody or antigen-binding fragment thereof that cross-competes with the antibody or antigen-binding fragment thereof as described herein.

In one aspect, the disclosure provides a method of making an antibody that specifically binds to an antigen. The method involves exposing the animal as described herein to the antigen; obtaining the sequence of (e.g. by sequencing) nucleic acids encoding human heavy chain immunoglobulin variable regions in a cell that expresses a chimeric heavy chain antibody that specifically binds to the antigen; and operably linking in a cell the nucleic acid encoding the human heavy chain immunoglobulin variable region with a nucleic acid encoding a human heavy chain immunoglobulin constant region.

The disclosure also relates to an offspring of the non-human mammal. In some embodiments, the non-human mammal is a rodent. In some embodiments, the non-human mammal is a mouse.

The disclosure also provides to a cell including the targeting vector as described herein. The disclosure also relates to a cell (e.g., a stem cell, an embryonic stem cell, an immune cell, a B cell, a T cell, or a hybridoma) or a cell line, or a primary cell culture thereof derived from the non-human mammal or an offspring thereof. The disclosure further relates to the tissue, organ or a culture thereof derived from the non-human mammal or an offspring thereof.

The disclosure further relates to the use of the non-human mammal or an offspring thereof, the animal model generated through the method as described herein in the development of a product related to an immunization process, the manufacture of a human antibody, or the model system for research in pharmacology, immunology, microbiology and medicine.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 lists CDR sequences of heavy chain variable region of anti-TFR1 antibodies according to Kabat numbering.

FIG. 2 lists CDR sequences of heavy chain variable region of anti-TFR1 antibodies according to IMGT numbering.

FIG. 3 lists amino acids sequences discussed in the disclosure.

FIG. 4A shows the antibody concentration in total brain protein of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 4B shows the ratio of antibody concentration in brain total protein to serum antibody concentration of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 4C shows the antibody concentration in brain parenchyma of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 4D shows the ratio of antibody concentration in brain parenchyma to serum antibody concentration of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 5A shows the antibody concentration test results in brain parenchyma after 24 hours of intravenous (i.v.) administration of hIgG1 (G1), JR141-N (G2), 23B8-N (G3), 24A1-N (G4), or 24G5-N (G5).

FIG. 5B shows the antibody concentration test results in brain total protein (Whole Brain) after 24 hours of intravenous (i.v.) administration of hIgG1 (G1), JR141-N (G2), 23B8-N (G3), 24A1-N (G4), or 24G5-N (G5).

FIG. 6 shows the antibody concentration results after 6 hours or 24 hours of intravenous (i.v.) administration of JR141-N (G2-G4) or 24G5-N (G5-G7)). hIgG1 was used as a negative control.

FIG. 7 shows the ADC concentration test results in brain parenchyma after 72 hours of intravenous (i.v.) administration of 24G5-ADC or 24G5-mono-ADC.

### DETAILED DESCRIPTION

A heavy-chain antibody (or heavy chain-only antibody) is an antibody which has only heavy chains (generally two heavy chains) and lacks the two light chains usually found in antibodies. Naturally occurring heavy-chain antibodies have been discovered in cartilaginous fishes (e.g., shark) and camelids (e.g., llama). For example, in cartilaginous fishes, the immunoglobulin new antigen receptor (IgNAR) is a heavy-chain antibody. IgNAR shows significant structural differences to other antibodies. It has five constant domains (CH) per chain instead of the usual three, several disulfide bonds in unusual positions, and the complementarity-determining region 3 (CDR3) forms an extended loop covering the site which binds to a light chain in other antibodies. These differences, in combination with the phylogenetic age of the cartilaginous fishes, have led to the hypothesis that IgNAR could be more closely related to a primordial antigen-binding protein than the mammalian immunoglobulins.

The only mammals with heavy-chain (IgG-like) antibodies are camelids such as dromedaries, camels, llamas and alpacas. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain (e.g., VHH) at their N-terminus. Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these heavy-chain antibodies (designated VHH, sdAb, or nanobody) is the smallest antigen-binding domain generated by adaptive immune systems. The Complementarity Determining Region 3 (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allows the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies, VHH do not display an intrinsic tendency to bind to light chains. This facilitates the induction of heavy chain antibodies in the presence of a functional light chain loci. Further, since VHH do not bind to VL domains, it is much easier to reformat VHHs into bispecific antibody constructs than constructs containing conventional VH-VL pairs or single domains based on VH domains.

A notable difference between the camelid VHH and the human VH domain is the length and orientation of the CDR3 loop. The CDR3 corresponds to the unique region of the antibody molecule that is encoded by a DNA element newly generated during B-cell development. Genetic recombination results in the fusion of a D-element with flanking V- and J-elements. During recombination further genetic diversity is generated by addition and/or deletion of nucleotides at the junctions. Thereby, the CDR3 loop provides the major contribution to antibody diversity and specificity. A limited number of variable region genes (IGHV, IGHD, and IGHJ) in some early transgenic heavy chain antibody animals, results in some antigens not being recognized by these animals, despite potent antigen response by wildtype animals (Janssens, Rick, et al. “Generation of heavy-chain-only antibodies in mice.” Proceedings of the National Academy of Sciences 103.41 (2006): 15130-15135). The present disclosure provides fully humanized heavy chain antibodies generated by genetically modified animals that have complete human heavy chain antibody repertoires.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, heavy chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, and Fv fragments.

As used herein, the term “human antibody” refers to an antibody that is encoded by a nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a mouse) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a human antibody and the constant domains of a non-human antibody. Additional examples of chimeric antibodies are described herein and are known in the art.

As used herein, the term “humanized antibody” refers to a non-human antibody which contains sequence derived from a non-human (e.g., mouse) immunoglobulin and contains sequences derived from a human immunoglobulin.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen.

As used herein, the term “heavy-chain antibody” refers to an antibody molecule which is composed only of heavy chains (generally two) and does not have any light chains.

As used herein, the term “VHH” refers to the variable domain derived from a heavy-chain antibody. The VHH can specifically recognize an antigen without the need to be paired with a VL. In some embodiments, the VHH (also know as sdAb or nanobody) described herein is derived from any of the humanized heavy-chain antibody described herein. In some embodiments, the VHH, sdAb, or nanobody described herein is derived from the heavy chain antibody produced by any of the genetically modified non-human animal described herein.

As used herein, the terms “subject” and “patient” are used interchangeably throughout the specification and describe an animal, human or non-human. Veterinary and non-veterinary applications are contemplated by the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

### Antibodies and Antigen Binding Fragments

The present disclosure provides antibodies and antigen-binding fragments thereof (e.g., heavy chain antibodies, humanized heavy chain antibodies, or multi-specific antibodies) that are produced by the methods described herein.

In general, conventional antibodies are made up of two classes of polypeptide chains, light chains and heavy chains. A non-limiting antibody of the present disclosure can be an intact, four-immunoglobulin-chain antibody comprising two heavy chains and two light chains. The heavy chain of the antibody can be of any isotype including IgM, IgG, IgE, IgA, or IgD or subclasses including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68(1-3): 9-16 (October 1997); Morea et al., J Mol Biol. 275(2): 269-94 (January 1998); Chothia et al., Nature 342(6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG2c, IgG3, IgG4, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety. The heavy chain constant regions in the heavy chain antibodies can be derived from any immunoglobulin molecules described herein (e.g., IgG1, IgG2a, IgG2b, IgG2c, IgG3, IgG4, IgM, IgD, IgE, IgA).

The antibody can also be an immunoglobulin molecule that is derived from any species (e.g., human, rodent, mouse, rat, camelid). Antibodies disclosed herein also include, but are not limited to, polyclonal, monoclonal, monospecific, multi-specific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of VHH as described herein, fused to CD3-zeta transmembrane- and endodomain.

The antibodies and antigen-binding fragments thereof (e.g., humanized antibodies or chimeric antibodies) that are produced by the methods described herein have various advantages. In some embodiments, no further optimization is required to obtain desired properties (e.g., binding affinities, thermal stabilities, and/or limited aggregation).

In some implementations, the antibody (or antigen-binding fragments thereof) specifically binds to a target with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) are greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) are less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. In some embodiments, the antibody binds to a target with KD less than or equal to about 0.9 nM, 0.8 nM, 0.7 nM, 0.6 nM, 0.5 nM, 0.4 nM, 0.3 nM, 0.2 nM, or 0.1 nM.

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C.

In various embodiments, substitutions are performed to a parental heavy chain antibody sequence to make a variant heavy chain antibody. In general, a heavy chain antibody variant of a parental heavy chain antibody has an antigen binding affinity that is at least 10%, at least 20%, at least 30%, at least 40%, at least 50%, at least 60%, at least 70%, at least 80%, at least 90% or at least 100% (e.g., at least 150%, at least 200%, at least 500%, at least 1000%, or up to at least 10,000%) of the binding affinity of the parental heavy chain antibody to a particular antigen. In some embodiments, a variant heavy chain antibody will comprise a single substitution as compared to a parental heavy chain antibody. However, in other embodiments, several amino acids, e.g., up to about 5 or 10 or more, are substituted as compared to the parental heavy chain antibody sequence that are derived from other human heavy chain sequences that share identity at a given position. In various embodiments, the resultant variant heavy chain antibody is tested to confirm that the desired binding affinity and/or specificity has not been significantly decreased by the replacement residues. In some embodiments, an improved variant heavy chain antibody is produced by the substitution of amino acids from a different human heavy chain sequence. In various embodiments, the VHH is at least 70%, 75%, 80%, 85%, 90%, 91%, 92%, 93%, 94%, 95%, 96%, 97%, 98%, 99%, or 100% identical to a parental VHH.

The VHH described herein can be used to make multi-specific (e.g., bispecific antibodies). In one aspect, the present disclosure provides a multi-specific antibody comprising: a first antigen binding portion and a second antigen binding portion. In some embodiments, the first antigen binding portion comprises a heavy chain variable domain (VH) and a light chain variable domain (VL), wherein the VH and VL together form an antigen-binding site that specifically binds a first epitope. In some embodiments, the first antigen binding portion comprises a VHH that specifically binds a first epitope. In some embodiments, the second antigen binding portion comprises a VHH that specifically binds a second epitope. In some embodiments, the first epitope and the second epitope are from the same antigen. In some embodiments, the first epitope and the second epitope are from different antigens.

In some embodiments, the first antigen binding portion is a full-length antibody consisting of two heavy chains and two light chains. In some embodiments, the first antigen binding portion is an antibody fragment comprising a heavy chain comprising the VH and a light chain comprising the VL. In some embodiments, the second antigen binding portion comprises a single polypeptide chain. In some embodiments, the C terminus of the second antigen binding portion is fused to the N-terminus of at least one heavy chain of the first antigen binding portion. In some embodiments, the C terminus of the second antigen binding portion is fused to the N-terminus of at least one light chain of the first antigen binding portion. In some embodiments, the N terminus of the second antigen binding portion is fused to the C-terminus of at least one heavy chain of the first antigen binding portion. In some embodiments, the N terminus of the second antigen binding portion is fused to the C-terminus of at least one light chain of the first antigen binding portion. In some embodiments, the second antigen binding portion is a Fab-like domain comprising a first polypeptide chain comprising a first VHH fused to a CH1 domain, and a second polypeptide chain comprising a second VHH fused to a CL domain.

In some embodiments, the antibody or antigen-binding fragment thereof is a tri-specific antibody. In some embodiments, the tri-specific antibody is a tri-specific VHH-Fc. In some embodiments, the tri-specific antibody comprises the same VHHs. In some embodiments, the tri-specific antibody comprises different VHHs. In some embodiments, the VHHs bind to the same epitope. In some embodiments, the VHHs bind to different epitopes.

In some embodiments, the antibody or antigen-binding fragment thereof has four or more than four VHHs. In some embodiments, in order to increase developability, at least four VHHs are combined without the addition of IgG Fc domain to construct tetra-specific VHHs. These molecules would have the added advantage of increased affinity and avidity towards the antigen compared to bi- and tri-specific VHH-Fcs, despite lacking the Fc effector functions.

In some embodiments, these the antibody or antigen-binding fragment thereof (e.g., comprising 1, 2, 3, 4, 5, 6, 7, 8, 9, 10, or more than 10) has a functional Fc.

In some embodiments, the heavy-chain antibody produced by the genetically modified non-human animal described herein has a VHH domain that includes CDR1, CDR2, and CDR3. In some embodiments, the CDR3 length is between 6-23, e.g., 6, 7, 8, 9, 10, 11, 12, 13, 14, 15, 16, 17, 18, 19, 20, 21, 22 or 23. In some embodiments, the CDR3 length is at least 6, at least 7, at least 8, at least 9, at least 10, at least 11, at least 12, at least 13, at least 14, at least 15, at least 16, at least 17, at least 18, at least 19, at least 20, at least 21, at least 22, or at least 23.

### Transferrin Receptor 1 (TFR1)

TFR1, also known as cluster of differentiation 71 (CD71), is widely expressed and can bind to transferrin (Tf) with high affinity. Human TFR1 is a 90 kDa type II transmembrane glycoprotein consisting of 760 amino acids that is found as a dimer (180 kDa) linked by disulfide bonds on the cell surface. The TFR1 monomer is composed of a large extracellular, C-terminal domain of 671 amino acids containing the Tf-binding site, a transmembrane domain (28 amino acids), and an intracellular N-terminal domain (61 amino acids). The C-terminal extracellular domain contains three N-linked glycosylation sites at asparagine residues 251, 317, and 727 and one O-linked glycosylation site at threonine 104, which are all required for adequate function of the receptor.

Transferrin (Tf) It is an 80 kDa glycoprotein composed of two 40 kDa subunits, known as the N- and C-lobes that are separated by a short linker sequence. Each subunit is capable of binding to one free ferric iron (Fe³⁺) and thus, Tf may have up to two atoms of iron attached. Tf in its iron free form, apo-Tf, binds Fe³⁺ with high efficiency in the blood and transports it to the cell surface for internalization through the interaction with TFR1. As a membrane protein regulating iron import, TFR1 is a member of the TFR family that shows nanomolar affinity to transferrin (Tf) bound to Fe³⁺. The complex of Tf-TFR1 is internalized through endocytosis mediated by clathrin, and Fe³⁺ is disassociated from Tf when pH decreases to 5.5. At this pH, apo-Tf and TFR1 are still associated and recycled to cell surface with physiological pH, so the former is released.

Iron uptake by transferrin receptor is an important way for cancer cells to absorb iron, thus accumulating evidence has proven that TFR1 participated in tumor onset and progression, and its expression was dysregulated significantly in many cancers. The relationship between TFR1 and cancers has been revealed, rendering TFR1 a valuable pharmaceutical target for intervening with cancers.

TFR1 expressed on the endothelial cells of the blood-brain barrier is used also in preclinical research to allow the delivery of large molecules including antibodies into the brain. The TFR1 targeting antibodies can cross the blood-brain barrier, without interfering with the uptake of iron.

A detailed description of TFR1, Tf, and their functions can be found, e.g., in Candelaria, P. V., et al. “Antibodies targeting the transferrin receptor 1 (TfR1) as direct anti-cancer agents.” Frontiers in Immunology 12 (2021): 607692; and Shen, Y., et al. “Transferrin receptor 1 in cancer: a new sight for cancer therapy.” American Journal of Cancer Research 8.6 (2018): 916; each of which is incorporated by reference in its entirety.

### Heavy Chain Single Variable Domain (VHH) Antibodies

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the immunoglobulin binding proteins.

The single variable domain of these antibodies (designated VHH, sdAb, or nanobody) is the smallest antigen-binding domain generated by adaptive immune systems. The third Complementarity Determining Region (CDR3) of the variable region of these antibodies has been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface.

VHHs offer numerous other advantages compared to conventional antibodies carrying variable domains (VH and VL) of conventional antibodies, including higher stability, solubility, expression yields, and refolding capacity, as well as better in vivo tissue penetration and internalization. Moreover, in contrast to the VH domains of conventional antibodies, VHH do not display an intrinsic tendency to bind to light chains. Since VHH do not bind to VL domains, it is much easier to reformat VHHs into multi-specific (e.g., bispecific antibody) constructs than constructs containing conventional VH-VL pairs or single domains based on VH domains.

The disclosure provides e.g., anti-TFR1 antibodies, the modified antibodies thereof, the chimeric antibodies thereof, and the humanized antibodies thereof.

The CDR sequences for 23B8, and 23B8 derived antibodies (e.g., humanized antibodies) include CDRs of the VHH domain as set forth in SEQ ID NOs: 1, 2, and 3, respectively, as defined by Kabat numbering. The CDRs can also be defined by IMGT system. Under the IMGT numbering, the CDRs of the VHH domain are set forth in SEQ ID NOs: 13, 14, and 15, respectively.

The CDR sequences for 24A1, and 24A1 derived antibodies (e.g., humanized antibodies) include CDRs of the VHH domain as set forth in SEQ ID NOs: 4, 5, and 6, respectively, as defined by Kabat numbering. The CDRs can also be defined by IMGT system. Under the IMGT numbering, the CDRs of the VHH domain are set forth in SEQ ID NOs: 16, 17, and 18, respectively.

The CDR sequences for 24C9, and 24C9 derived antibodies (e.g., humanized antibodies) include CDRs of the VHH domain as set forth in SEQ ID NOs: 7, 8, and 9, respectively, as defined by Kabat numbering. The CDRs can also be defined by IMGT system. Under the IMGT numbering, the CDRs of the VHH domain are set forth in SEQ ID NOs: 19, 20, and 21, respectively.

The CDR sequences for 24G5, and 24G5 derived antibodies (e.g., humanized antibodies) include CDRs of the VHH domain as set forth in SEQ ID NOs: 10, 11, and 12, respectively, as defined by Kabat numbering. The CDRs can also be defined by IMGT system. Under the IMGT numbering, the CDRs of the VHH domain are set forth in SEQ ID NOs: 22, 23, and 24, respectively.

The amino acid sequence for the VHH domain of 23B8 antibody is set forth in SEQ ID NO: 25. The amino acid sequence for the VHH domain of 24A1 antibody is set forth in SEQ ID NO: 26. The amino acid sequence for the VHH domain of 24C9 antibody is set forth in SEQ ID NO: 27. The amino acid sequence for the VHH domain of 24G5 antibody is set forth in SEQ ID NO: 28.

The amino acid sequences for various modified or humanized VHH are also provided. As there are different ways to modify or humanize a heavy-chain antibody (e.g., a sequence can be modified with different amino acid substitutions), the VHH domain of a heavy-chain antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOS: 25-28.

Furthermore, in some embodiments, the antibodies or antigen-binding fragments thereof described herein can also contain one, two, or three VHH domain CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, and SEQ ID NOs: 22-24.

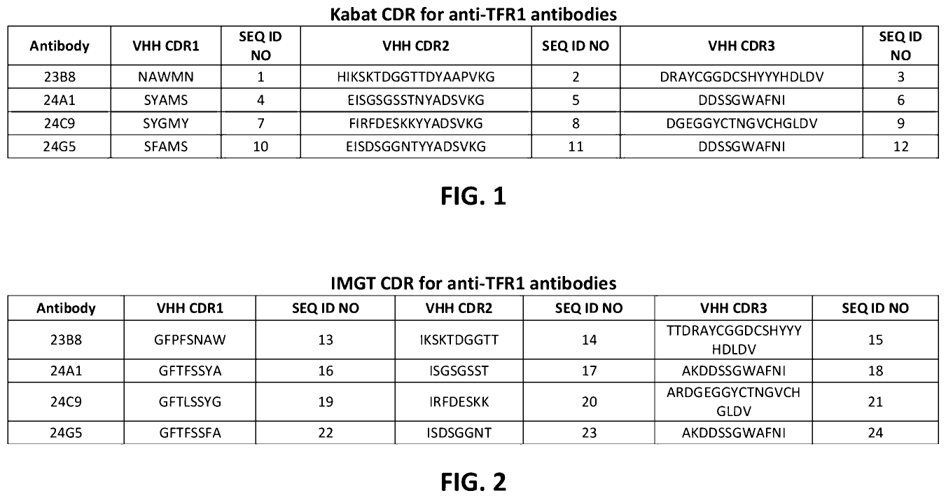

In some embodiments, the antibodies can have a heavy chain single variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences are shown in FIG. 1 and FIG. 2.

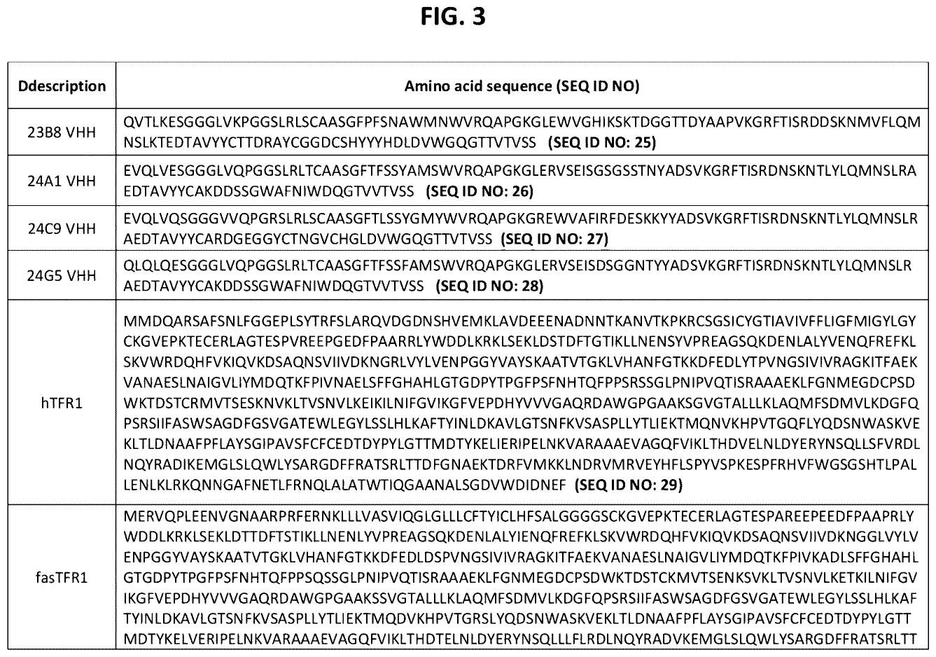

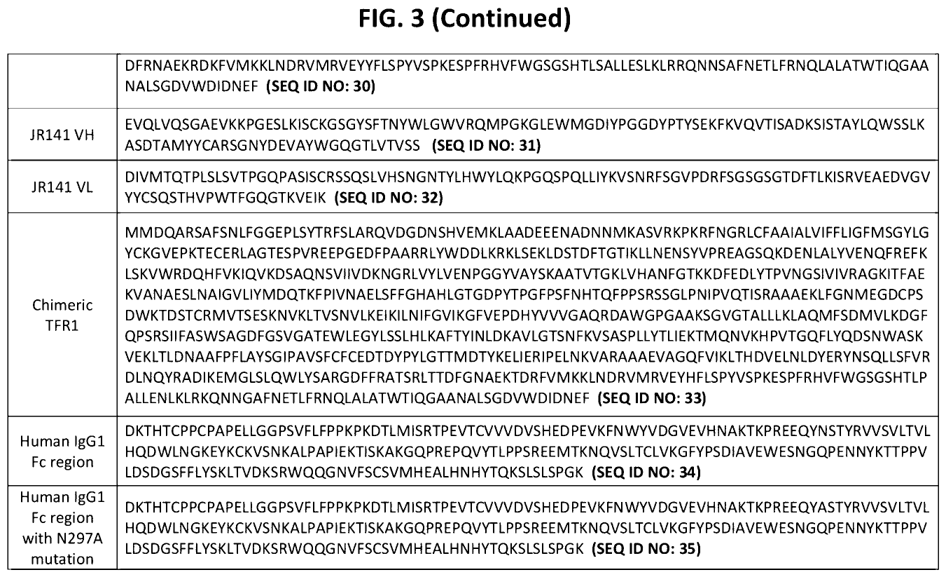

In some embodiments, the antibody or an antigen-binding fragment described herein can contain a heavy chain single variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 3.

In some embodiments, the antibody or an antigen-binding fragment described herein can contain a heavy chain single variable domain (VHH) containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme. In some embodiments, the CDR is determined based on IMGT numbering scheme.

The disclosure also provides antibodies or antigen-binding fragments thereof that bind to TFR1 (human TFR1). The antibodies or antigen-binding fragments thereof contain a heavy chain single variable region (VHH) comprising or consisting of an amino acid sequence that is at least 80%, 85%, 90%, or 95% identical to a selected VHH sequence. In some embodiments, the selected VHH sequence is SEQ ID NO: 25. In some embodiments, the selected VHH sequence is SEQ ID NO: 26. In some embodiments, the selected VHH sequence is SEQ ID NO: 27. In some embodiments, the selected VHH sequence is SEQ ID NO: 28.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5. The disclosure also provides nucleic acid comprising a polynucleotide encoding a polypeptide comprising an immunoglobulin heavy chain single variable domain (VHH). The VHH comprises CDRs as shown in FIG. 1 and FIG. 2, or has sequences as shown in FIG. 3.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

In some embodiments, the antibody or antigen-binding fragment thereof comprises a heavy chain single variable domain (VHH) CDR1 selected from SEQ ID NOs: 1, 4, 7, 10, 13, 16, 19, and 22.

In some embodiments, the antibody or antigen-binding fragment thereof comprises a heavy chain single variable domain (VHH) CDR2 selected from SEQ ID NOs: 2, 5, 8, 11, 14, 17, 20, and 23.

In some embodiments, the antibody or antigen-binding fragment thereof comprises a heavy chain single variable domain (VHH) CDR3 selected from SEQ ID NOs: 3, 6, 9, 12, 15, 18, 21, and 24.

### Antibody Characteristics

TFR1 performs a critical role in cellular iron uptake through the interaction with iron-bound TF. Iron is required for multiple cellular processes and is essential for DNA synthesis and, thus, cellular proliferation. Due to its central role in cancer cell pathology, malignant cells often overexpress TFR1 and this increased expression can be associated with poor prognosis in different types of cancer. The elevated levels of TfR1 expression on malignant cells, together with its extracellular accessibility, ability to internalize, and central role in cancer cell pathology make this receptor an attractive target for antibody-mediated therapy.

In some embodiments, the antibodies or antigen-binding fragments thereof described herein cannot block the binding between TFR1 and TF. In some embodiments, the antibodies or antigen-binding fragments thereof described herein can block the binding between TFR1 and TF. In some embodiments, the antibodies or antigen-binding fragments thereof described herein can be conjugated to anti-cancer agents that are internalized by receptor-mediated endocytosis. In some embodiments, the antibodies or antigen-binding fragments thereof described herein can disrupt the function of the receptor. In some embodiments, the antibodies or antigen-binding fragments thereof described herein cannot induce Fc effector functions, therefore preventing or ameliorating their negative effects to normal cells.

The disclosure provides antibodies or antigen-binding fragments thereof comprising a human Fc domain, which induce Fc-dependent effector functions by at least or about at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof comprising a human Fc domain, which induce host immune response by at least or about at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof that can internalize into human brain cells (e.g., cortical microvascular endothelial cells) that the endocytosis rate is at least 50%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%. In some embodiments, the endocytosis rate of the antibodies or antigen-binding fragments thereof described herein is at least 1-fold, 2-fold, 3-fold, 4-fold, 5-fold, 6-fold, 7-fold, 8-fold, 9-fold, 10-fold, 50-fold, 100-fold, 500-fold, or 1000-fold as compared to that of an isotype control antibody.

In some embodiments, provided herein is an antibody or antigen-binding fragment thereof comprising a single heavy chain. In some embodiments, provided herein is an antibody or antigen-binding fragment thereof comprising a pair of heavy chains. In some embodiments, the heavy chain pair is linked by disulfide bonds. In some embodiments, the heavy chain pair comprises knob-in-hole modifications. In some embodiments, the heavy chain comprises a human IgG Fc domain. In some embodiments, the antibody or antigen-binding fragment thereof comprises in each heavy chain at least 1, 2, 3, 4, 5, 6, 7, 8, 9, or 10 VHH domains. In some embodiments, the VHH domains in each heavy chain specifically bind to the same epitope. In some embodiments, the VHH domains in each heavy chain specifically bind to different epitopes. In some embodiments, the VHH domains in each heavy chain bind to at least 1, 2, 3, 4, or 5 different epitopes.

In some embodiments, the antibody or antigen-binding fragment thereof is a bi-specific antibody, or tri-specific antibody. In some embodiments, the antibody or antigen-binding fragment thereof can specifically bind to at least 4, 5, or 6 antigens.

In some embodiments, the antibody (or antigen-binding fragment thereof) specifically binds to TFR1 with a dissociation rate (koff) of less than 0.1 s⁻¹, less than 0.01 s⁻¹, less than 0.001 s⁻¹ 1, less than 0.0001 s⁻¹ 1, or less than 0.00001 s⁻¹. In some embodiments, the dissociation rate (koff) is greater than 0.01 s⁻¹ 1, greater than 0.001 s⁻¹ 1, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to human TFR1, monkey TFR1, mouse TFR1, or chimeric TFR1. In some embodiments, the antibody does not bind to human TFR1, monkey TFR1, mouse TFR1, or chimeric TFR1.

In some embodiments, thermal stabilities are determined. The antibodies or antigen-binding fragments as described herein can have a Tm (melting temperature) greater than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C. In some embodiments, Tm is less than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C. The antibodies or antigen-binding fragments as described herein can have a Tagg (aggregation temperature, e.g., Tagg at 266 nm (Tagg266) or Tagg at 473 nm (Tagg473)) great than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C. In some embodiments, Tagg is less than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C.

In some embodiments, the Fc region is human IgG1, human IgG2, human IgG3, or human IgG4.

In some embodiments, the antibodies or antigen binding fragments thereof have a functional Fc region. In some embodiments, the antibodies or antigen binding fragments thereof comprise a human IgG1 Fc region. In some embodiments, the human IgG1 Fc region comprises an amino acid sequence that is at least 80%, 85%, 90%, 95%, 96%, 97%, 98%, 99%, or 100% identical to SEQ ID NO: 34.

In some embodiments, the antibodies or antigen binding fragments do not have an Fc region. For example, the antibody (or antigen-binding fragment thereof) is a polypeptide comprising one or more VHH domains that are interconnected by linker peptides. In some embodiments, the antibody comprises at least 1, 2, 3, 4, 5, 6, 7, 8, 9, or 10 VHH domain. In some embodiments, the VHH domains specifically bind to the same epitope. In some embodiments, the VHH domains bind to different epitopes. In some embodiments, the VHH domains bind to at least 1, 2, 3, 4, or 5 different epitopes.

In some embodiments, the antibodies or antigen binding fragments thereof do not have a functional Fc region. In some embodiments, the Fc region has LALA mutations (L234A and L235A mutations in EU numbering), or LALA-PG mutations (L234A, L235A, P329G mutations in EU numbering). In some embodiments, the Fc region has a mutation at position 297 (e.g., N297A) according to EU numbering. In some embodiments, the mutated human IgG1 Fc region comprises an amino acid sequence that is at least 80%, 85%, 90%, 95%, 96%, 97%, 98%, 99%, or 100% identical to SEQ ID NO: 35.

In some embodiments, after being administered to a subject after 6 hours, 12 hours, 24 hours, 36 hours, 48 hours, 60 hours, or 72 hours, concentration of the antibody or antigen binding fragment thereof described herein in brain (e.g., whole brain or parenchyma) can be greater than 10%, 20%, 30%, 40%, 50%, 60%, 70%, or 80% of its concentration immediately (e.g., 0.5 hour) after administration. In some embodiments, after being administered to a subject after 6 hours, 12 hours, 24 hours, 36 hours, 48 hours, 60 hours, or 72 hours, concentration of the antibody or antigen binding fragment thereof described herein in brain (e.g., whole brain or parenchyma) can be at least 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 5000-fold, or 10000-fold of the concentration of a control antibody (e.g., hIgG1 or JR141-N), or the concentration in serum of the subject.

### Methods of Making Anti-TFR1 Antibodies

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., TFR1. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites. In some embodiments, the heavy-chain antibody or antigen-binding fragment thereof described herein is obtained by immunizing any of the genetically modified animals (e.g., mice with complete human heavy chain variable domain substitution in situ, combined with a modified constant region) described e.g., in PCT/CN2022/119188.

Humanized antibodies include antibodies having variable and constant regions derived from (or having the same amino acid sequence as those derived from) human germline immunoglobulin sequences of human immunoglobulin scaffold sequences. Humanized antibodies may include amino acid residues not encoded by human germline immunoglobulin sequences (e.g., mutations introduced by random or site-specific mutagenesis in vitro or by somatic mutation in vivo). Accordingly, “humanized” antibodies are chimeric antibodies wherein sequences from a non-human species are substituted by the corresponding human sequences.

Ordinarily, amino acid sequence variants of the human, humanized, or chimeric anti-TFR1 antibody will contain an amino acid sequence having at least 75%, 80%, 85%, 90%, 95%, 96%, 97%, 98%, or 99% percent identity with a sequence present in the VHH domain of the original antibody.

Identity or homology with respect to an original sequence is usually the percentage of amino acid residues present within the candidate sequence that are identical with a sequence present within the human, humanized, or chimeric anti-TFR1 antibody or fragment, after aligning the sequences and introducing gaps, if necessary, to achieve the maximum percent sequence identity, and not considering any conservative substitutions as part of the sequence identity.

Additional modifications to the anti-TFR1 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-TFR1 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be affected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods in Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

In some embodiments, the antibody or antigen-binding fragment thereof is expressed in yeast, insect cells, or mammalian cells (e.g., CHO cells).

### Methods of Treatment and Diagnosis

The anti-TFR1 antibodies or antibody or antigen-binding fragments thereof of the present disclosure can be used for various therapeutic purposes. In one aspect, the disclosure provides methods for treating a brain disease (e.g., brain cancer, dementia, or Alzheimer's disease) in a subject, methods of identifying a subject having a brain disease (e.g., brain cancer, dementia, or Alzheimer's disease), methods of reducing the risk of developing a brain disease, or methods of reducing the risk of developing additional symptoms in a subject. In some embodiments, the treatment can halt, slow, retard, or inhibit progression of a brain disease (e.g., brain cancer, dementia, or Alzheimer's disease). In some embodiments, the treatment can result in the reduction of in the number, severity, and/or duration of one or more symptoms of a brain disease (e.g., brain cancer, dementia, or Alzheimer's disease) in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a brain disease).

In one aspect, the disclosure features methods that carry a therapeutic agent to cross the blood brain barrier. In some embodiments, the antibodies or antigen-binding fragments thereof as described herein are linked to the therapeutic agent. In some embodiments, the therapeutic agent is an antibody, an antigen binding fragment thereof, a small molecule, or an antibody-drug conjugate.

In some embodiments, the compositions and methods disclosed herein can be used for treatment of patients at risk for a brain disease (e.g., brain cancer, dementia, or Alzheimer's disease). Patients with a brain disease (e.g., brain cancer, dementia, or Alzheimer's disease) can be identified with various methods known in the art.

In some embodiments, the brain disease is a brain cancer.

In one aspect, the disclosure is related to methods of decreasing the rate of tumor growth, including contacting a tumor cell with an effective amount of a composition including the antibody or antigen-binding fragment thereof, or the antibody-drug conjugate described herein. In one aspect, the disclosure is related to methods of killing a tumor cell, including contacting a tumor cell with an effective amount of a composition including the antibody or antigen-binding fragment thereof, or the antibody-drug conjugate described herein.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment thereof is an amount sufficient to ameliorate, stop, stabilize, reverse, inhibit, slow and/or delay progression of a disease in a patient. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of the disease). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIR1 are administered to the subject.

In some embodiments, the additional therapeutic agent is an anti-PD-1 antibody, an anti-PD-L1 antibody, an anti-LAG-3 antibody, an anti-TIGIT antibody, an anti-4-1BB antibody, an anti-CTLA-4 antibody, an anti-CD40 antibody, an anti-OX40 antibody, or an anti-GITR antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject in a subject, or a subject identified as being at risk of developing the disease, decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Human Anti-TFR1 Antibodies

RenNano™ mice (Biocytogen, complete human heavy chain variable domain substitution in situ, combined with a modified constant region. The mice are described e.g., in PCT/CN2022/119188, which is incorporated herein by reference in its entirety) were immunized with His-tagged human TFR1 (transferrin receptor 1) protein (hTFR1-His, ACROBiosystems, Cat #: CD1-H5243) to obtain anti-TFR1 antibodies. Before immunization, retro-orbital blood was collected as a negative control. Freund's complete adjuvant (CFA) was used for the first immunization and Freund's incomplete adjuvant (IFA) was used for the second and third immunizations. A total of three immunizations (bi-weekly) were performed. One week after the third immunization, retro-orbital blood was collected, and the antibody titer of serum was detected by FACS.

Procedures to enhance immunization were also performed at least fourteen days after the previous immunizations. TFR1 protein was injected by intraperitoneal injection, and the CHO-S cells expressing human TFR1 antigen was injected through the tail vein.

Antigen-specific immune cells were isolated from the immunized mice to further obtain anti-TFR1 antibodies or to obtain the heavy chain variable region sequences of the anti-TFR1 antibodies. For example, single cell technology (e.g., using Beacon® Optofluidic System, Berkeley Lights Inc.) was used to screen and find plasma cells that secrete antigen-specific monoclonal antibodies. Reverse transcription and PCR sequencing were used to obtain antibody variable region sequences. The obtained variable region sequences were used for antibody expression to verify the binding activity to TFR1 using FACS. Because the lack of the CH1 domain, the heavy chain variable region (VH) of the obtained antibodies is also referred to as a heavy chain single variable domain (VHH).

Specifically, the obtained VHH sequences were respectively connected to a human IgG1 constant region (e.g., the hinge region, CH2 domain and CH3 domain). Exemplary antibodies obtained by this method included: 23B8, 24A1, 24C9 and 24G5. The heavy chain CDR1-3 sequences are shown in FIG. 1 and FIG. 2. The VHH region sequences of 23B8, 24A1, 24C9 and 24G5 are shown in FIG. 3.

The constant region of the antibodies can be further engineered to replace the Asparagine at position 297 with Alanine (N297A). For example, when N297A mutation is introduced into the constant region of 24G5, the resulting antibody is named as 24G5-N.

Further, one-armed antibodies 23B8-mono, 24A1-mono, 24C9-mono and 24G5-mono were constructed, these antibodies have an anti-TFR1 arm comprising a VHH region, and a heavy chain fragment comprising CH2 and CH3 domains of IgG1 with N297A mutation.

### Example 2. Cross-Species Binding of Anti-TFR1 Antibodies

CHO-S-hTFR1 cells or CHO-S-fasTFR1 cells were transferred to a 96-well plate at a density of 10⁵ cells/well respectively. Serially diluted sample anti-TFR1 antibodies were added to the 96-well plate, and incubated at 4 °C for 30 minutes. PBS was used as a negative control (NC). Then, the cells were incubated with the secondary antibody anti-hIgG-Fc-Alex Flour™ 647 (Jackson ImmunoResearch Laboratories, Cat #: 109-606-170) at 4 °C in the dark for 15 minutes before flow cytometry analysis.

CHO-S-hTFR1 cells or CHO-S-fasTFR1 cells were obtained by transfecting CHO-S cells with vectors expressing human TFR1 (hTFR1, SEQ ID NO: 29) or Macaca fascicularis (crab-eating macaque) TFR1 amino acid sequence (fasTFR1, SEQ ID NO: 30), respectively. The test results are shown in the table below.

JR141, a humanized IgG1 antibody targeting human TFR1 conjugated to human iduronate-2-sulfatase, was first approved in March 2021 in Japan for the intravenous treatment of mucopolysaccharidosis type II. The VH and VL sequences of JR141 are set forth in SEQ ID NO: 31 and SEQ ID NO: 32, respectively. For positive control (JR141-N), the VH and VL of JR141 were connected to a human IgG1 constant region with N297A mutation.

TABLE 1

| | | Percentage of | |
|---|---|---|---|
| Antibody | Cells | Positive Cells | Evaluation |
| NC | CHO-S-hTFR1 | 0.1% | No Binding |
| | CHO-S-fasTFR1 | 0.2% | No Binding |
| JR141-N | CHO-S-hTFR1 | 45.0% | Binding |
| | CHO-S-fasTFR1 | 37.2% | Binding |
| 23B8 | CHO-S-hTFR1 | 38.8% | Binding |
| | CHO-S-fasTFR1 | 39.6% | Binding |
| 24A1 | CHO-S-hTFR1 | 56.4% | Binding |
| | CHO-S-fasTFR1 | 50.9% | Binding |
| 24C9 | CHO-S-hTFR1 | 45.8% | Binding |
| | CHO-S-fasTFR1 | 0.35% | No Binding |
| 24G5 | CHO-S-hTFR1 | 54.3% | Binding |
| | CHO-S-fasTFR1 | 51.2% | Binding |

### Example 3. Binding Affinity of Anti-TFR1 Antibodies

The binding affinity of the anti-TFR1 antibodies to His-tagged TFR1 protein of human (hTFR1-His, ACROBiosystems, Cat #: CD1-H5243) or monkey (fasTFR1-His, ACROBiosystems, Cat #: TFR-C524a) were verified using surface plasmon resonance (SPR) on Biacore™ (Biacore, Inc., Piscataway N.J.) 8K biosensor equipped with pre-immobilized Protein A sensor chips.

Purified anti-TFR1 antibodies was captured on the Protein A chip (Series S Sensor Chip Protein A) for the detection. Purified anti-TFR1 antibodies (1 μg/mL) was loaded at 10 μL/min to bind to hTFR1-His and fasTFR1-His (200 nM). The flow rate was 30 μL/min. The binding and dissociation time were set to 180 seconds and 600 seconds, respectively. The chip was regenerated after the last injection of each titration with a glycine solution (pH 2.0) at 30 μL/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below. The results show that all four anti-TFR1 antibodies can bind to human and monkey TFR1 with high affinity.

TABLE 2

| Protein | Analyte 1 Solution | kon (1/Ms) | koff (1/s) | KD (M) |
|---|---|---|---|---|
| 23B8 | hTFR1-His | 1.51E+04 | 1.32E−04 | 8.73E−09 |
| | fasTFR1-His | 4.10E+05 | 7.99E−03 | 1.95E−08 |
| 24A1 | hTFR1-His | 2.62E+04 | 7.68E−05 | 2.93E−09 |
| | fasTFR1-His | 4.36E+04 | 1.01E−04 | 2.32E−09 |
| 24C9 | hTFR1-His | 2.68E+04 | 3.83E−04 | 1.43E−08 |
| | fasTFR1-His | 2.78E+04 | 3.68E−04 | 1.32E−08 |
| 24G5 | hTFR1-His | 4.24E+04 | 8.68E−05 | 2.05E−09 |
| | fasTFR1-His | 6.33E+04 | 8.79E−05 | 1.39E−09 |

### Example 4. Epitope Analysis of Anti-TFR1 Antibodies

Relative positions of target protein epitope between a pair of purified anti-TFR1 antibodies were analyzed by Biolayer Interferometry (BLI) using ForteBio Octet system at 30 °C. 1×HBS-EP+ buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH7.4) diluted from HBS-EP+ buffer (10×) was used as the running buffer throughout the experiment. About 10 μg/mL of hTFR1-His protein was captured by HIS1K (Anti-Penta-HIS) for 200 seconds, and 200 nM of antibody (analyte 1) was injected at a flow rate of 30 μL/min to bind the ligand. Another antibody (analyte 2) was injected under the same conditions to determine whether the binding of different antibodies interfered with each other. The binding time was 300 seconds for each antibody.

The binding value of each antibody was obtained using Data Analysis HT 12.0. To quantify the interference of one antibody binding to another, a binding ratio was calculated to compare each pair of antibodies. The binding ratio is defined as the binding value of the second antibody (analyte 2), divided by the binding value of the first antibody (analyte 1). The binding ratio of each antibody pair is summarized in the matrix table below. Specifically, the binding ratio was between 0.0 to 0.5, if analyte 1 exhibited a blocking effect to analyte 2; the binding ratio was between 0.5-1.1, if analyte 1 did not exhibit a blocking effect to analyte 2. In general, antibody pairs that interfere with each other have the same or overlapping epitopes.

The epitope binding assay results indicate that 24A1 and 24G5 can recognize the same epitope, and 23B8, 24C9 and JR141-N can recognize different epitopes.

TABLE 3

Analyte 1

| Analyte 2 | 23B8 | 24A1 | 24G5 | 24C9 | JR141-N |
|---|---|---|---|---|---|
| 23B8 | 0.1 | 0.2 | 0.3 | 1.4 | 1.1 |
| 24A1 | 0.0 | 0.1 | 0.1 | 0.1 | 0.3 |
| 24G5 | 0.0 | 0.0 | 0.0 | 0.0 | 0.2 |
| 24C9 | 0.6 | 0.1 | 0.2 | 0.0 | 0.8 |
| JR141-N | 0.7 | 0.0 | 0.0 | 1.1 | 0.0 |

### Example 5. Internalization of Anti-TFR1 Antibodies

Anti-TFR1 antibodies together with the pHAb-Goat anti-human IgG secondary antibody were added to human cortical microvascular endothelial cells (hCMEC/D3 cells), and incubated for 3 hours. After incubation, the cells were centrifuged and washed with FACS buffer. Mean fluorescence intensity (MFI) was measured using a flow cytometer. Endocytosis rates of antibodies were calculated. Human IgG1 protein (CrownBio, Cat #: C0001) was used as an isotype control (ISO). The results are shown in the following table, indicating that all four antibodies exhibited good endocytosis activity in human cortical microvascular endothelial cells.

TABLE 4

| Antibody | MFI | Positive Population |
|---|---|---|
| ISO | 4288 | 0.9% |
| 23B8 | 11392 | 67.0% |
| 24A1 | 26972 | 96.6% |
| 24C9 | 37086 | 96.0% |
| 24G5 | 26406 | 96.0% |

### Example 6. Developability Analysis of Anti-TFR1 Antibodies

Developability of anti-TFR1 antibodies 23B8, 24A1, 24C9 and 24G5 was evaluated. The antibodies were diluted to 1 mg/mL with water. Specifically, the following tests were performed: (1) observing the solution appearance and presence of visible non-soluble objects; (2) detecting The Purity Changes of Antibodies by Size-Exclusion Ultra Performance Liquid Chromatography (SEC-UPLC) (indicated as the percentage of the main peak area to the sum of all peak areas (Purity, %)); (3) detecting changes in the apparent hydrophobicity of the antibodies using the Hydrophobic Interaction Chromatography-High Performance Liquid Chromatography (HIC-HPLC) method (indicated as the retention time of the main peak (HIC, min); (4) detecting charge variants in the antibodies by Capillary Isoelectric Focusing (cIEF) (indicated as the percentages of the main component, acidic component, and alkaline component); and (5) detecting the thermal stability of antibodies via the UNcle system (indicated as the melting temperature (Tm) and aggregation temperature (Tagg)).

In the SEC-UPLC experiments, the Agilent 1290 chromatograph system (connected with XBridge™ Protein BEH SEC column (200 Å, Waters Corporation)) was used. The antibody samples were diluted to 1 mg/mL with purified water. The following parameters were used: mobile phase: 25 mM phosphate buffer (PB) (pH 6.8)+0.3 M NaCl; flow rate: 1.8 mL/min; column temperature: 25 °C; detection wavelength: 280 nm; injection volume: 10 μL; sample tray temperature: 6 °C; and running time: 7 minutes.

In the HIC-HPLC experiments, the Agilent 1260 chromatograph system (connected with ProPac™ HIC-10 column (4.6×100 mm, Thermo Scientific)) was used, and samples were diluted using mobile phase A to 0.5 mg/mL. The following parameters were used: mobile phase A: 0.9 M ammonium sulfate, 0.1 M PB, 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30 °C; detection wavelength: 280 nm; injection volume: 10 μg; sample tray temperature: about 6 °C; and running time: 45 minutes.

In the cIEF experiments, the Maurice cIEF Method Development Kit (Protein Simple, Cat #: PS-MDK01-C) was used for sample preparation. Specifically, 40 μg protein sample was mixed with the following reagents in the kit: 1 μL Maurice cIEF pI Marker-4.05, 1 μL Maurice cIEF pI Marker-9.99, 35 μL 1% Methyl Cellulose Solution, 2 μL Maurice cIEF 500 mM Arginine, 4 μL Ampholytes (Pharmalyte pH ranges 3-10), and water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes. The analysis software installed on the instrument was used to analyze the absorbance of the 280 nm-focused protein.

In the thermal stability experiments, antibody solutions at 60 mg/mL were heated from 25 °C to 95 °C using 1 °C increments, with an equilibration time of 1 minute before each measurement.

Furthermore, the antibodies were diluted to 1 mg/mL using PBS buffer, and the following tests were performed: (1) detecting the specificity of the antibodies using the Cross-Interaction Chromatography (CIC) method (indicated as the retention time (CIC, min)); (2) detecting the colloidal stabilities of the antibodies by the stand-up monolayer chromatography (SMAC) method (indicated as the retention time (SMAC, %/min)).

In the CIC assay, a CIC column was prepared by coupling human polyclonal IgGs (Sigma, Cat #: 14506) onto a HiTrap NHS-activated resin (GE Healthcare, Cat #: 17-0716-01) followed by passivation with ethanolamine according to published procedures. The column was then connected to Agilent 1260 chromatograph system and run at 0.1 mL/min using 1×PBS as the mobile phase until a flat baseline was reached. 10 μg of antibodies at 1 mg/mL in PBS were then injected. Peak retention times on the column were monitored at 280 nm; running time: 50 minutes.

In the SMAC assay, Zenix column (4.6 mm×30 cm, Sepax, Cat #: 213300-4630) was connected to the column compartment, and place the appropriate line in the mobile phase. Equilibrate column for 60 min at 0.350 mL/min flow rate with mobile phase buffer. Load antibodies into the injection sequence. Mobile Phase A: 150 mM Sodium Phosphate pH 7.0; flow rate: 0.35 mL/min; running time: 25 min; column temperature: 30 °C; detection wavelength: 280 nm, 220 nm.

Detailed results are summarized in the table below.

TABLE 5

| | SEC | | | | | Tm | | | |
|---|---|---|---|---|---|---|---|---|---|
| | Purity | HIC | | CIC | SMAC | Tm1 | Tm2 | Tm3 | Tagg |
| Antibody | (%) | (min) | PI | (min) | (%/min) | (°C) | (°C) | (°C) | (°C) |
| 23B8 | 92.82 | 19.909 | 7.376 | 9.317 | 98.15/9.399 | 58.87 | 67.80 | 84.13 | 56.48 |
| 24A1 | 98.74 | 2.486 | 6.795 | 8.699 | 89.71/8.824 | 64.80 | 73.80 | 82.20 | 52.73 |
| 24C9 | 98.20 | 3.740 | 8.595 | 9.960 | 96.67/8.851 | 65.38 | 69.90 | 78.20 | 33.63 |
| 24G5 | 98.86 | 3.829 | 6.495 | 8.786 | 95.69/8.862 | 60.86 | 77.66 | 85.30 | 76.03 |

For the Tagg of 24C9, a slight fluctuation was observed at 33.63 °C from the Tagg curve (data not shown), while massive aggregation occured at 50-60 °C. To confirm the thermal stability, SEC-UPLC of 24C9 was tested after treatment of 40 °C for 14 days. After heating, the purity of 24C9 stayed above 97%. These results indicated that all the four antibodies have good developabilities.

### Example 7. Pharmacokinetics (PK) Analysis

A humanized TFR1 mouse model (hTFR1 mice) was engineered to express a chimeric TFR1 protein (SEQ ID NO: 33) in which the extracellular region of mouse TFR1 protein was replaced with the corresponding human TFR1 extracellular region. A detailed description regarding the humanized TFR1 mouse model can be found in PCT Application No. PCT/CN2022/105924, which is incorporated herein by reference in its entirety.

The concentrations of the anti-TFR1 antibodies were determined in hTFR1 mice. Specifically, the mice were placed into different groups (8 mice per group), and administered with an approximately equal molar dosage of JR141-N (G2), 23B8-N (G3), 24A1-N (G4), 24G5-N (G5), or 24C9-N (G6), by intravenous (i.v.) injection. The control group (G1) mice were administered with human IgG1 (hIgG1). Details of the administration scheme are shown in the table below.

TABLE 6

| Group | No. of mice | Antibodies | Dosage | Route | Frequency |
|---|---|---|---|---|---|
| G1 | 8 | hIgG1 | 18.4 mg/kg | i.v. | single dose |
| G2 | 8 | JR141-N | 18.4 mg/kg | i.v. | single dose |
| G3 | 8 | 23B8-N | 10.0 mg/kg | i.v. | single dose |
| G4 | 8 | 24A1-N | 10.0 mg/kg | i.v. | single dose |
| G5 | 8 | 24G5-N | 10.0 mg/kg | i.v. | single dose |
| G6 | 8 | 24C9-N | 10.0 mg/kg | i.v. | single dose |

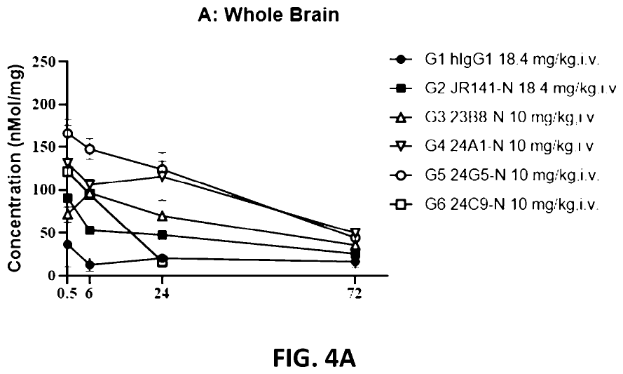

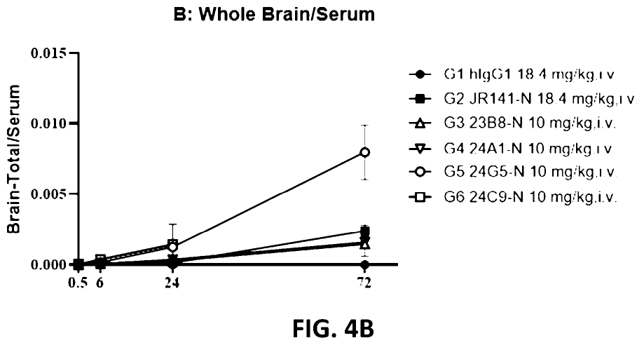

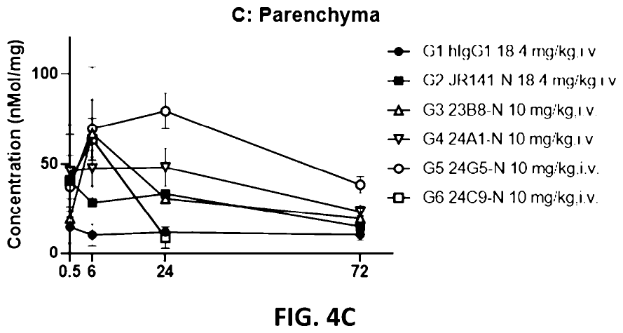

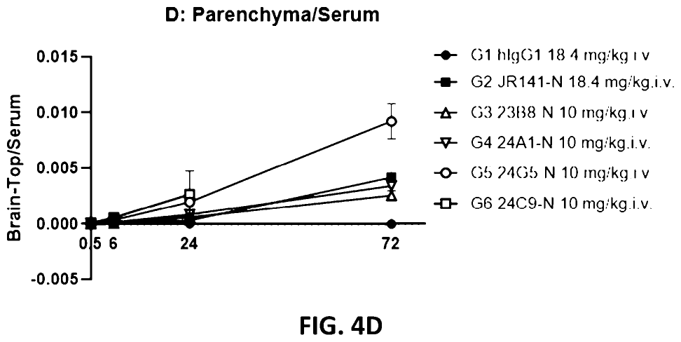

Blood and brain samples were collected 0.5, 6, 24, and 72 hours after the administration. Two mice were sampled at each time point, and the mice were anesthetized after retro-orbital blood was collected. To avoid interference from the residual blood in the brain, the mice were perfused by saline for 10 minutes at room temperature. Specifically, saline was perfused via systemic circulation from the left ventricle to the right ventricle. Brain samples were excised and divided into two hemibrains by the sagittal plane. The left hemibrain was subjected to quantification of the injected antibody, while the right hemibrain was fixed by formalin and embedded with paraffin for serial sections. The brain samples were cut into pieces and homogenized with DPBS (Dulbecco's phosphate-buffered saline) containing 1× mixed protease inhibitors. The brain homogenate was aliquoted for protein extraction followed by antibody quantification by electrochemiluminescence. For the rest homogenate, capillaries were depleted by gradient density centrifugation at 5400 g for 15 minutes, using 15% dextran. After centrifugation, the fraction at the top of the centrifuge tube was saved as parenchyma and subjected to protein extraction and antibody quantification as well. FIGS. 4A-4D show the antibody concentration in total brain protein (FIG. 4A), the ratio of antibody concentration in brain total protein to serum antibody concentration (FIG. 4B), the antibody concentration in brain parenchyma (FIG. 4C), and the ratio of antibody concentration in brain parenchyma to serum antibody concentration at each time point (FIG. 4D). These results demonstrated that 24G5-N (group G5) was the most abundant either in parenchyma or in the whole brain.

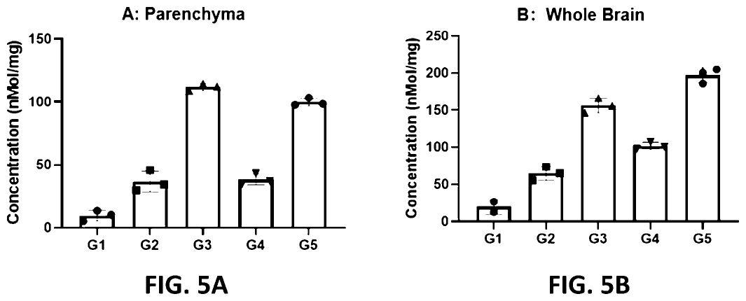

In a similar experiment, hTFR1 mice were placed into five groups (3 mice per group) and administered with 18.4 mg/kg JR141-N (G2), 10 mg/kg 23B8-N (G3), 10 mg/kg 24A1-N (G4), or 10 mg/kg 24G5-N (G5) by intravenous injection (1 administration in total). The control group (G1) mice were administered with hIgG1 (G1). 24 hours after administration, brain samples were collected to determine the concentrations of the anti-TFR1 antibodies. FIGS. 5A-5B show the antibody concentration test results in brain parenchyma and brain total protein, respectively. All tested antibodies showed a higher concentration in brain than hIgG1 (G1), indicating that compared with the positive control JR141-N (G2), 23B8-N (G3) and 24G5-N (G5) can better pass across the blood-brain barrier and enter the brain parenchyma.

In another similar experiment, hTFR1 mice were placed into seven groups (6 mice per group) and administered with JR141-N (G2-G4) or 24G5-N (G5-G7)) by intravenous (i.v.) injection. The control group (G1) mice were administered with hIgG1. Details of the administration scheme are shown in the table below.

TABLE 7

| | | | Dosage | | |
|---|---|---|---|---|---|
| Group | No. of mice | Antibodies | (mg/kg) | Route | Frequency |
| G1 | 6 | hIgG1 | 5.52 mg/kg | i.v. | single dose |
| G2 | 6 | JR141-N | 1.84 mg/kg | i.v. | single dose |
| G3 | 6 | JR141-N | 5.52 mg/kg | i.v. | single dose |
| G4 | 6 | JR141-N | 18.4 mg/kg | i.v. | single dose |
| G5 | 6 | 24G5-N | 1 mg/kg | i.v. | single dose |
| G6 | 6 | 24G5-N | 3 mg/kg | i.v. | single dose |
| G7 | 6 | 24G5-N | 10 mg/kg | i.v. | single dose |

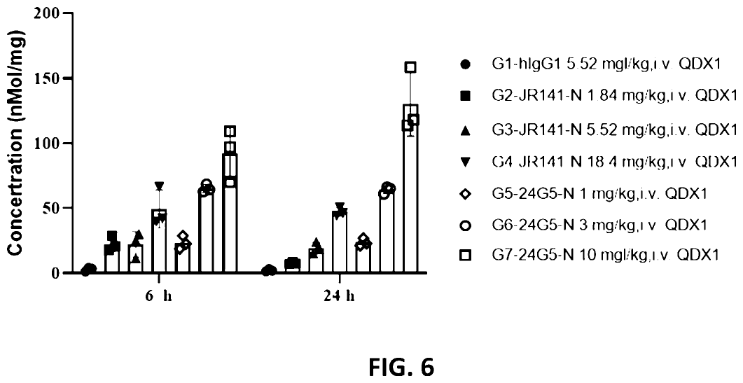

6 hours and 24 hours after the administration, blood and brain samples were collected using the methods described above. Three mice were sampled at each time point. Treatment of the tissues and quantification of antibodies were also performed as described above. The measurement results of the concentration of humanized anti-TFR1 antibodies in the brain parenchyma are shown in FIG. 6. The results show that the antibody concentration accumulated in the brain parenchyma of 24G5-N was significantly higher than that of hIgG1 under each dosage condition. Furthermore, the concentration of both JR141-N and 24G5-N exhibited a dose-dependent trend in brain parenchyma.

To detect the distribution of the humanized anti-TFR1 antibody 24G5-N in mouse brain, immunofluorescence assays were performed by staining hIgG, hTFR1, and mCD31, respectively, on right hemibrain sections of the mice used in the experiment above. The results showed that, mCD31 was well-labeled in the microvessels. hTFR1 was also detected on microvessels, which was co-localized with mCD31. Besides, hTFR1 expression was also detected on several neurons in parenchyma. In particular, the anti-TFR1 antibody 24G5-N was stained by a secondary anti-IgG antibody conjugated with DyLight® 488. Similar to hTFR1, 24G5-N was detected in microvessels and parenchyma, and its signal overlapped with the hTFR1 signal. Thus, either for the quantification of 24G5-N in the whole brain or parenchyma, or the visual evidence of immunofluorescence of 24G5-N in the parenchyma, the results indicate that anti-TFR1 antibody 24G5-N can pass across the blood-brain barrier (BBB) efficiently.

### Example 8. Blocking Assay

Blockade of TFR1 binding to TF (transferrin) by anti-TFR1 antibodies 23B8, 24A1, 24C9 and 24G5 were tested by Biolayer Interferometry (BLI) using ForteBio Octet® system at 30 °C. Specifically, 1×HBS-EP+ buffer (10 mM 4-(2-hydroxyethyl)-1-piperazineethanesulfonic acid (HEPES), 150 mM NaCl, 3 mM EDTA and 0.05% Surfactant P20, pH 7.4) diluted from HBS-EP+ buffer (10×) was used as the running buffer throughout the experiment. About 10 μg/mL of antibodies were captured by AHC (Anti-Human IgG Fc Capture) for 200 seconds, and 800 nM of hTFR1-His (ACROBiosystems, Cat #: CD1-H5243) and hTF-His (human transferrin protein, Kactus Biosystems, Cat #: TFN-HM101) were injected to bind the ligand. The binding time was 300 seconds for each antibody. The binding value of each antibody was obtained using Data Analysis HT 12.0. The results showed that these four antibodies did not block the binding of TFR1 to TF. Therefore, such non-blocking antibodies are not likely to interfere with TFR1-TF interactions in normal cells.

### Example 9. Antibody Drug Conjugates (ADC)

Each purified antibody (23B8-N, 24A1-N, 24C9-N, 24G5-N, 23B8-mono, 24A1-mono, 24C9-mono and 24G5-mono) was coupled with Dxd (Deruxtecan) through GGFG linker.

For the names of antibody drug conjugates, “ADC” is added directly after the antibody name. For example, if 24G5-N is coupled to GGFG-Dxd, it is named as 24G5-ADC. For another examples, if 24G5-mono is coupled to GGFG-Dxd, it is named as 24G5-mono-ADC. HIC-HPLC (Reversed Phase High Performance Liquid Chromatography) was used to detect the coupling of antibodies with drug molecules. The HIC-HPLC detection results showed that the drug-to-antibody ratio (DAR) of the ADCs was about 3.

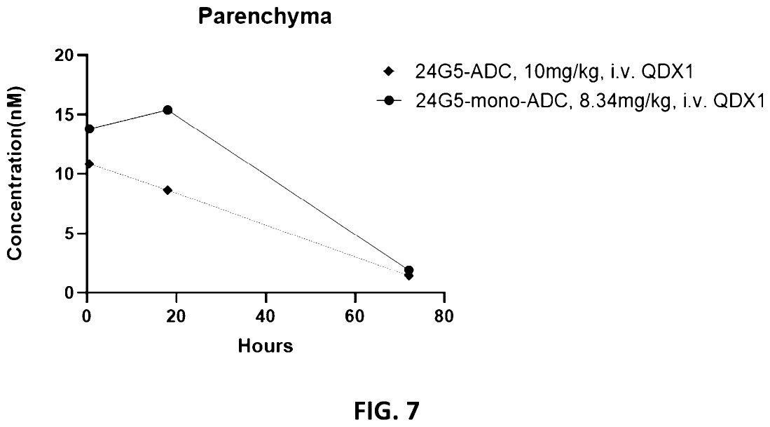

hTFR1 mice were placed into different groups (6 mice per group) and administered with 10 mg/kg bivalent anti-TFR1 ADCs (24G5-ADC, 23B8-ADC, 24A1-ADC, 24C9-ADC) or the same molar dose 8.34 mg/kg one-armed monovalent anti-TFR1 ADCs (23B8-mono-ADC, 24A1-mono-ADC, 24C9-mono-ADC, or 24G5-mono-ADC) by intravenous (i.v.) injection (1 administration in total). 0.5 hour, 18 hours and 72 hours after the administration, blood and brain samples were collected using the methods described in Example 7. Treatment of the tissues and quantification of antibodies and ADCs were also performed as described above The results indicated that all the tested ADCs are able to bring Dxd across the BBB efficiently. Exemplary results are shown in FIG. 7, 24G5-ADC and 24G5-mono-ADC bring Dxd across the BBB, of which the monovalent 24G5-mono-ADC has a better penetration effect than the bivalent format 24G5-ADC.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to transferrin receptor 1 (TFR1), comprising:
a heavy chain single variable domain (VHH) comprising complementarity determining regions (CDRs) 1, 2, and 3, wherein the VHH CDR1 region comprises an amino acid sequence that is at least 80%, 85%, 90%, 95%, 96%, 97%, 98%, 99%, or 100% identical to a selected VHH CDR1 amino acid sequence, the VHH CDR2 region comprises an amino acid sequence that is at least 80%, 85%, 90%, 95%, 96%, 97%, 98%, 99%, or 100% identical to a selected VHH CDR2 amino acid sequence, and the VHH CDR3 region comprises an amino acid sequence that is at least 80%, 85%, 90%, 95%, 96%, 97%, 98%, 99%, or 100% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively;
(4) the selected VHH CDRs 1, 2, 3 amino acid sequences are set forth in SEQ ID NOs: 10, 11, and 12, respectively;
(5) the selected VHH CDRs 1, 2, 3 amino acid sequences are set forth in SEQ ID NOs: 13, 14, and 15, respectively;
(6) the selected VHH CDRs 1, 2, 3 amino acid sequences are set forth in SEQ ID NOs: 16, 17, and 18, respectively;
(7) the selected VHH CDRs 1, 2, 3 amino acid sequences are set forth in SEQ ID NOs: 19, 20, and 21, respectively; and
(8) the selected VHH CDRs 1, 2, 3 amino acid sequences are set forth in SEQ ID NOs: 22, 23, and 24, respectively.

2. The antibody or antigen-binding fragment thereof of claim 1, wherein the VHH comprises CDRs 1, 2, 3 with the amino acid sequences set forth in SEQ ID NOs: 1, 2, and 3, respectively.

3. The antibody or antigen-binding fragment thereof of claim 1, wherein the VHH comprises CDRs 1, 2, 3 with the amino acid sequences set forth in SEQ ID NOs: 4, 5, and 6, respectively.

4. The antibody or antigen-binding fragment thereof of claim 1, wherein the VHH comprises CDRs 1, 2, 3 with the amino acid sequences set forth in SEQ ID NOs: 7, 8, and 9, respectively.

5. The antibody or antigen-binding fragment thereof of claim 1, wherein the VHH comprises CDRs 1, 2, 3 with the amino acid sequences set forth in SEQ ID NOs: 10, 11, and 12, respectively.

6. An antibody or antigen-binding fragment thereof that binds to TFR1 comprising a heavy chain single variable region (VHH) comprising an amino acid sequence that is at least 80%, 85%, 90%, 95%, 96%, 97%, 98%, 99%, or 100% identical to a selected VHH sequence, wherein the selected VHH sequence is selected from the group consisting of SEQ ID NOs: 25, 26, 27, and 28.

7. The antibody or antigen-binding fragment thereof of claim 6, wherein the VHH comprises the sequence of SEQ ID NO: 25.

8. The antibody or antigen-binding fragment thereof of claim 6, wherein the VHH comprises the sequence of SEQ ID NO: 26.

9. The antibody or antigen-binding fragment thereof of claim 6, wherein the VHH comprises the sequence of SEQ ID NO: 27.

10. The antibody or antigen-binding fragment thereof of claim 6, wherein the VHH comprises the sequence of SEQ ID NO: 28.

11. The antibody or antigen-binding fragment thereof of any one of claims 1-10, wherein the antibody or antigen-binding fragment specifically binds to a human TFR1, a monkey TFR1, a mouse TFR1, or a chimeric TFR1.

12. The antibody or antigen-binding fragment thereof of any one of claims 1-11, wherein the antibody or antigen-binding fragment is a human or humanized antibody or antigen-binding fragment thereof.

13. The antibody or antigen-binding fragment thereof of any one of claims 1-12, wherein the antibody or antigen-binding fragment is a multi-specific antibody (e.g., a bispecific antibody).

14. An antibody or antigen-binding fragment thereof comprising the VHH CDRs 1, 2, 3, of the antibody or antigen-binding fragment thereof of any one of claims 1-13.

15. The antibody or antigen-binding fragment thereof of any one of claims 1-14, wherein the antibody or antigen-binding fragment comprises a human IgG Fc (e.g., a human IgG1 Fc).

16. The antibody or antigen-binding fragment thereof of claim 15, wherein the human IgG Fc comprises a non-asparagine residue (e.g., alanine) at position 297 according to EU numbering.

17. The antibody or antigen-binding fragment thereof of any one of claims 1-16, wherein the antibody or antigen-binding fragment comprises two or more heavy chain single variable domains.

18. A nucleic acid comprising a polynucleotide encoding the antibody or antigen-binding fragment thereof any one of claims 1-17.

19. The nucleic acid of claim 18, wherein the nucleic acid is cDNA.

20. A vector comprising one or more of the nucleic acids of claim 18 or 19.

21. A cell comprising the vector of claim 20.

22. The cell of claim 21, wherein the cell is a CHO cell.

23. A cell comprising one or more of the nucleic acids of claim 18 or 19.

24. A method of producing an antibody or an antigen-binding fragment thereof, the method comprising
(a) culturing the cell of any one of claims 21-23 under conditions sufficient for the cell to produce the antibody or the antigen-binding fragment thereof; and
(b) collecting the antibody or the antigen-binding fragment thereof produced by the cell.

25. An antibody-drug conjugate comprising the antibody or antigen-binding fragment thereof of any one of claims 1-17 covalently bound to a therapeutic agent.

26. The antibody drug conjugate of claim 25, wherein the therapeutic agent is a cytotoxic or cytostatic agent.

27. A method of treating a subject having a brain disease (e.g., a brain cancer), the method comprising administering a therapeutically effective amount of a composition comprising the antibody or antigen-binding fragment thereof of any one of claims 1-17, or the antibody-drug conjugate of claim 25 or 26, to the subject.

28. The method of claim 27, wherein the antibody or antigen-binding fragment thereof, or the antibody-drug conjugate can pass across the blood-brain barrier (BBB) of the subject.

29. A method of treating a subject having a cancer, the method comprising administering a therapeutically effective amount of a composition comprising the antibody or antigen-binding fragment thereof of any one of claims 1-17, or the antibody-drug conjugate of claim 25 or 26, to the subject.

30. The method of claim 29, wherein the cancer is brain cancer, lung cancer, gastric cancer, colorectal cancer, liver cancer, ovarian cancer, prostate cancer, leukemia, or breast cancer.

31. A method of identifying a subject as having a brain disease (e.g., a brain cancer), the method comprising
detecting a sample collected from the subject as having the brain disease by the antibody or antigen-binding fragment thereof of any one of claims 1-17,
thereby identifying the subject as having the brain disease.

32. The method of claim 31, wherein the sample is a brain parenchyma sample from the subject.

33. The method of any one of claims 27-32, wherein the subject is a human subject.

34. A method of delivering an agent to cross blood brain barrier, the method comprising
administering the agent covalently linked to the antibody or antigen-binding fragment thereof of any one of claims 1-17 to the subject.

35. The method of claim 34, wherein the agent is an antibody or an antibody drug conjugate.

36. The method of claim 34 or 35, wherein the agent is anti-amyloid antibody.

37. A pharmaceutical composition comprising the antibody or antigen-binding fragment thereof of any one of claims 1-17, and a pharmaceutically acceptable carrier.

38. A pharmaceutical composition comprising the antibody drug conjugate of claim 25 or 26, and a pharmaceutically acceptable carrier.

39. An antibody or antigen-binding fragment thereof that cross-competes with the antibody or antigen-binding fragment thereof of any one of claims 1-17.


SEQUENCE LISTING (35 sequences)

SEQ ID NO: 1 - protein, 5 aa, synthetic construct
NAWMN

SEQ ID NO: 2 - protein, 19 aa, synthetic construct
HIKSKTDGGTTDYAAPVKG

SEQ ID NO: 3 - protein, 19 aa, synthetic construct
DRAYCGGDCSHYYYHDLDV

SEQ ID NO: 4 - protein, 5 aa, synthetic construct
SYAMS

SEQ ID NO: 5 - protein, 17 aa, synthetic construct
EISGSGSSTNYADSVKG

SEQ ID NO: 6 - protein, 10 aa, synthetic construct
DDSSGWAFNI

SEQ ID NO: 7 - protein, 5 aa, synthetic construct
SYGMY

SEQ ID NO: 8 - protein, 17 aa, synthetic construct
FIRFDESKKYYADSVKG

SEQ ID NO: 9 - protein, 17 aa, synthetic construct
DGEGGYCTNGVCHGLDV

SEQ ID NO: 10 - protein, 5 aa, synthetic construct
SFAMS

SEQ ID NO: 11 - protein, 17 aa, synthetic construct
EISDSGGNTYYADSVKG

SEQ ID NO: 12 - protein, 10 aa, synthetic construct
DDSSGWAFNI

SEQ ID NO: 13 - protein, 8 aa, synthetic construct
GFPFSNAW

SEQ ID NO: 14 - protein, 10 aa, synthetic construct
IKSKTDGGTT

SEQ ID NO: 15 - protein, 21 aa, synthetic construct
TTDRAYCGGDCSHYYYHDLDV

SEQ ID NO: 16 - protein, 8 aa, synthetic construct
GFTFSSYA

SEQ ID NO: 17 - protein, 8 aa, synthetic construct
ISGSGSST

SEQ ID NO: 18 - protein, 12 aa, synthetic construct
AKDDSSGWAFNI

SEQ ID NO: 19 - protein, 8 aa, synthetic construct
GFTLSSYG

SEQ ID NO: 20 - protein, 8 aa, synthetic construct
IRFDESKK

SEQ ID NO: 21 - protein, 19 aa, synthetic construct
ARDGEGGYCTNGVCHGLDV

SEQ ID NO: 22 - protein, 8 aa, synthetic construct
GFTFSSFA

SEQ ID NO: 23 - protein, 8 aa, synthetic construct
ISDSGGNT

SEQ ID NO: 24 - protein, 12 aa, synthetic construct
AKDDSSGWAFNI

SEQ ID NO: 25 - protein, 130 aa, synthetic construct
QVTLKESGGGLVKPGGSLRLSCAASGFPFSNAWMNWVRQAPGKGLEWVGHIKSKTDGGTTDYAAPVKGRFTISRDDSKNMVFLQMNSLKTEDTAVYYCTTDRAYCGGDCSHYYYHDLDVWGQGTTVTVSS

SEQ ID NO: 26 - protein, 119 aa, synthetic construct
EVQLVESGGGLVQPGGSLRLTCAASGFTFSSYAMSWVRQAPGKGLERVSEISGSGSSTNYADSVKGRFTISRDNSKNTLYLQMNSLRAEDTAVYYCAKDDSSGWAFNIWDQGTVVTVSS

SEQ ID NO: 27 - protein, 126 aa, synthetic construct
EVQLVQSGGGVVQPGRSLRLSCAASGFTLSSYGMYWVRQAPGKGREWVAFIRFDESKKYYADSVKGRFTISRDNSKNTLYLQMNSLRAEDTAVYYCARDGEGGYCTNGVCHGLDVWGQGTTVTVSS

SEQ ID NO: 28 - protein, 119 aa, synthetic construct
QLQLQESGGGLVQPGGSLRLTCAASGFTFSSFAMSWVRQAPGKGLERVSEISDSGGNTYYADSVKGRFTISRDNSKNTLYLQMNSLRAEDTAVYYCAKDDSSGWAFNIWDQGTVVTVSS

SEQ ID NO: 29 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 30 - protein, 727 aa, Macaca fascicularis
MERVQPLEENVGNAARPRFERNKLLLVASVIQGLGLLLCFTYICLHFSALGGGGSCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 31 - protein, 118 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSS

SEQ ID NO: 32 - protein, 112 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 33 - protein, 760 aa, synthetic construct
MMDQARSAFSNLFGGEPLSYTRFSLARQVDGDNSHVEMKLAADEEENADNNMKASVRKPKRFNGRLCFAAIALVIFFLIGFMSGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 34 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK